(19) United States
(12) Patent Application Publication
(10) Pub. No.: US 2016/0353789 A1
(43) Pub. Date: Dec. 8, 2016

(54) NUTRITION COMPOSITION SUPPRESSING GROWTH OF PROTOZOAN PARASITES OF BLOOD CELLS

(71) Applicant: AJINOMOTO CO., INC., Tokyo (JP)

(72) Inventors: Makoto BANNAI, Kawasaki-shi (JP); Kenji Nagao, Kawasaki-shi (JP); Hirotaka Kanuka, Tokyo (JP)

(73) Assignee: AJINOMOTO CO., INC., Tokyo (JP)

(21) Appl. No.: 15/244,349

(22) Filed: Aug. 23, 2016

Related U.S. Application Data
(63) Continuation of application No. PCT/JP2015/056019, filed on Feb. 24, 2015.

(30) Foreign Application Priority Data
Feb. 24, 2014 (JP) 2014-032546

(51) Int. Cl. A23L 33/175; A61K 31/198; A61K 31/4172; A61K 31/405

(52) U.S. Cl. CPC A23L 33/175; A61K 31/405; A61K 31/198; A61K 31/4172; A23V 2002/00

(57) ABSTRACT

Compositions, comprising one or more amino acids selected from the group consisting of tryptophan, methionine, phenylalanine, valine, leucine, histidine, lysine and threonine, and isoleucine at a content of not more than 1.5 wt % relative to the total amount of the nutrition composition, provide a more economical and effective nutrition composition for suppressing the growth of hemocytozoon such as malaria parasite and the like.

### CROSS REFERENCES TO RELATED APPLICATIONS

This application is a continuation of International Patent Application No. PCT/JP15/056019, filed on Feb. 24, 2015, and claims priority to Japanese Patent Application No. 2014-032546, filed on Feb. 24, 2014, both of which are incorporated herein by reference in their entireties.

### BACKGROUND OF THE INVENTION

Field of the Invention

The present invention relates to nutrition compositions that suppress the growth of hemocytozoon such as malaria parasite and the like.

Discussion of the Background

Malaria is an infectious disease caused by malaria parasite that invades the body by anopheles bites. Many people are infected with the disease in the subtropical and tropical areas, and it is a highly important infectious disease worldwide. Malaria includes four kinds of falciparum malaria, vivax malaria, ovale malaria, and quartan fever malaria. Among these, falciparum malaria becomes severe in a short period and is sometimes lethal unless appropriately treated in an early stage. In addition, undernutrition induces severity of malaria and increases mortality rate.

For the treatment of malaria, classical medicaments such as quinine hydrochloride, chloroquine, mefloquine, a sulfadoxine/pyrimethamine combination agent and the like, and new antimalarial agents such as an atovaquone/proguanil combination agent, artemisinin medicaments (artemisinin, artesunate, artemether) and the like are used. To compensate for the disadvantageous short efficacy duration of the artemisinin medicaments, a treatment method using an artemisinin medicament in combination with other antimalarial agent, which is called ACT (artemisinin-based combination therapy), for example, an artemether/lumefantrine combination agent, an artesunate/amodiaquine combination agent and the like, is also adopted. Classical medicaments are economical but have the problems of side effects, drug resistance and the like. New antimalarial agents are highly effective and show relatively low side effects and drug resistance, but they are expensive and have problems of non-prevalence in malaria-infected areas and the like. Therefore, an effective treatment method of malaria, which is more economical and shows low side effects and drug resistance, is desired.

In the meantime, it has been reported that the metabolism of malaria parasite and the progress of developmental cycle become slow when isoleucine is removed from the medium of malaria parasite (Plasmodium falciparum) in the red blood cell (see Proc. Natl. Acad. Sci. USA. 2012 Nov. 20; 109(47):E3278-87, which is incorporated herein by reference in its entirety). In addition, thiaisoleucine, which is a derivative of isoleucine, has been studied as a target of the development of an antimalarial agent (see Proc. Natl. Acad. Sci. USA. 2011 Jan. 25; 108(4):1627-32, which is incorporated herein by reference in its entirety). However, it has not been reported heretofore that the intake of an isoleucine-deficient nutrition composition suppresses the growth of malaria parasite in the body. In addition, a nutrition composition deficient only in isoleucine, from among essential amino acids (tryptophan, methionine, phenylalanine, valine, leucine, histidine, lysine, threonine and isoleucine), has not been reported at all to date.

### SUMMARY OF THE INVENTION

Accordingly, it is one, object of the present invention to provide novel and more economical and effective nutrition compositions that suppress the growth of hemocytozoon such as malaria parasite and the like.

It is another object of the present invention to provide novel nutrition compositions that treat the infectious disease caused by hemocytozoon such as malaria and the like.

These and other objects, which will become apparent during the following detailed description, have been achieved by the inventors' discovery that a nutrition composition containing isoleucine in an amount not more than a certain level suppresses the growth of hemocytozoon, which resulted in the completion of the present invention.

Accordingly, the present invention provides the following:

(1) A nutrition composition for suppressing the growth of hemocytozoon, comprising one or more amino acids selected from the group consisting of tryptophan, methionine, phenylalanine, valine, leucine, histidine, lysine and threonine, and isoleucine at a content of not more than 1.5 wt % relative to the total amount of the nutrition composition.

(2) A nutrition composition for suppressing the growth of hemocytozoon, comprising one or more amino acids selected from the group consisting of tryptophan, methionine, phenylalanine, valine, leucine, histidine, lysine and threonine, and isoleucine at a content of not more than 3.8 wt % relative to the total amount of the amino acids.

(3) The nutrition composition of the above-mentioned (1) or (2), which does not contain isoleucine.

(4) The nutrition composition of any of the above-mentioned (1) to (3), further comprising one or more nutrients selected from the group consisting of carbohydrate, protein, fat, vitamin and mineral.

(5) A nutrition composition for the treatment of infectious disease caused by hemocytozoon, comprising one or more amino acids selected from the group consisting of tryptophan, methionine, phenylalanine, valine, leucine, histidine, lysine and threonine, and isoleucine at a content of not more than 1.5 wt % relative to the total amount of the nutrition composition.

(6) A nutrition composition for the treatment of infectious disease caused by hemocytozoon, comprising one or more amino acids selected from the group consisting of tryptophan, methionine, phenylalanine, valine, leucine, histidine, lysine and threonine, and isoleucine at a content of not more than 3.8 wt % relative to the total amount of the amino acids.

(7) The nutrition composition of the above-mentioned (5) or (6), which does not contain isoleucine.

(8) The nutrition composition of any one of the above-mentioned (5) to (7), further comprising one or more nutrients selected from the group consisting of carbohydrate, protein, fat, vitamin and mineral.

(9) A nutrition composition comprising tryptophan, methionine, phenylalanine, valine, leucine, histidine, lysine and threonine, and isoleucine at a content of not more than 1.5 wt % relative to the total amount of the nutrition composition.

(10) A nutrition composition comprising tryptophan, methionine, phenylalanine, valine, leucine, histidine, lysine and threonine, and isoleucine at a content of not more than 3.8 wt % relative to the total amount of the amino acids.

(11) The nutrition composition of the above-mentioned (9) or (10), which does not contain isoleucine.

(12) The nutrition composition of any of the above-mentioned (9) to (11), further comprising one or more nutrients selected from the group consisting of carbohydrate, protein, fat, vitamin and mineral.

(13) The nutrition composition of any of the above-mentioned (9) to (12), for suppressing the growth of hemocytozoon.

(14) The nutrition composition of any of the above-mentioned (1) to (4) and (13), wherein the hemocytozoon is a malaria parasite.

(15) The nutrition composition of any one of the above-mentioned (9) to (12), for treating the infectious disease caused by hemocytozoon.

(16) The nutrition composition of any one of the above-mentioned (5) to (8), wherein the infectious disease caused by hemocytozoon is a malaria.

(17) The nutrition composition of any one of the above-mentioned (5) to (8), (14) and (16), which is used in combination with other antimalarial agent(s).

(18) The nutrition composition of the above-mentioned (17), wherein said other antimalarial agent is one or more medicaments selected from the group consisting of quinine hydrochloride, chloroquine, mefloquine, a sulfadoxine/pyrimethamine combination agent, an atovaquone/proguanil combination agent, artemisinin, artesunate, artemether, an artemether/lumefantrine combination agent and an artesunate/amodiaquine combination agent.

(19) A method of suppressing the growth of hemocytozoon, comprising administering an effective amount of the nutrition composition of any of the above-mentioned (1) to (4) and (9) to (13) to a vertebrate.

(20) The method of the above-mentioned (19), wherein the hemocytozoon is a malaria parasite.

(21) A method of treating infectious disease caused by hemocytozoon, comprising administering an effective amount of the nutrition composition of any one of the above-mentioned (5) to (12) and (15) to a vertebrate.

(22) The method of the above-mentioned (21), wherein the hemocytozoon is a malaria parasite.

(23) The method of the above-mentioned (22), further comprising administering an effective amount of other antimalarial agent(s).

(24) The method of the above-mentioned (23), wherein said other antimalarial agent is one or more medicaments selected from the group consisting of quinine hydrochloride, chloroquine, mefloquine, a sulfadoxine/pyrimethamine combination agent, an atovaquone/proguanil combination agent, artemisinin, artesunate, artemether, an artemether/lumefantrine combination agent and an artesunate/amodiaquine combination agent.

According to the present invention, a nutrition composition that suppresses the growth of hemocytozoon such as a malaria parasite and the like can be provided. In addition, a nutrition composition for the treatment of infectious disease caused by hemocytozoon such as malaria can be provided. Furthermore, the nutrition composition of the present invention is superior in safety, since the ingredients thereof are amino acids and the like, which are less feared to cause side effects.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 shows a malaria parasite growth-inhibitory effect in BALB/c strain mice by the administration of an isoleucine deficient diet.

FIG. 2 shows a survival rate curve of malaria parasite-infected BALB/c strain mice administered with an isoleucine deficient diet.

FIG. 3 shows a malaria parasite growth-inhibitory effect in C3H/HeN strain mice by the administration of an isoleucine deficient diet.

FIG. 4 shows a survival rate curve of malaria parasite-infected C3H/HeN strain mice administered with an isoleucine deficient diet.

FIG. 5 shows a malaria parasite growth-inhibitory effect in C57BL6/J strain mice by the administration of an isoleucine deficient diet.

FIG. 6 shows a survival rate curve of malaria parasite-infected C57BL6/J strain mice administered with an isoleucine deficient diet.

FIG. 7 shows a Malaria parasite growth-inhibitory effect in BALB/c strain mice by the administration of a diet having stepwisely-decreased isoleucine contents.

FIG. 8 shows a malaria parasite growth-inhibitory effect in BALE/c strain mice by the administration of an isoleucine deficient diet after infection with a malaria parasite.

### DETAILED DESCRIPTION OF THE PREFERRED EMBODIMENTS

The embodiment of the present invention is explained in the following.

The nutrition composition of the present invention is a nutrition composition for suppressing the growth of hemocytozoon, comprising one or more amino acids selected from the group consisting of tryptophan, methionine, phenylalanine, valine, leucine, histidine, lysine and threonine, and isoleucine at a content of not more than 1.5 wt % relative to the total amount of the nutrition composition.

In another embodiment of the present invention, the nutrition composition of the present invention is a nutrition composition for suppressing the growth of hemocytozoon, comprising one or more amino acids selected from the group consisting of tryptophan, methionine, phenylalanine, valine, leucine, histidine, lysine and threonine, and isoleucine at a content of not more than 3.8 wt % relative to the total amount of the amino acids.

In another embodiment of the present invention, the nutrition composition of the present invention is a nutrition composition comprising tryptophan, methionine, phenylalanine, valine, leucine, histidine, lysine and threonine, and isoleucine at a content of not more than 1.5 wt % relative to the total amount of the nutrition composition.

In another embodiment of the present invention, the nutrition composition of the present invention is a nutrition composition comprising tryptophan, methionine, phenylalanine, valine, leucine, histidine, lysine and threonine, and isoleucine at a content of not more than 3.8 wt % relative to the total amount of the amino acids.

The nutrition composition of the present invention contains one or more amino acids selected from the group consisting of tryptophan, methionine, phenylalanine, valine, leucine, histidine, lysine and threonine, preferably, one or more amino acids selected from the group consisting of histidine, lysine and threonine. In another preferable embodiment, the nutrition composition of the present invention contains tryptophan, methionine, phenylalanine, valine, leucine, histidine, lysine and threonine.

In the present invention, it is important that the content of isoleucine in the nutrition composition is not more than a certain level. The content of isoleucine in the nutrition composition can be appropriately selected depending on a usual daily intake of isoleucine for a variety of vertebrates.

The content of isoleucine in the nutrition composition of the present invention is not more than 1.5 wt % relative to the total amount of the nutrition composition, preferably not more than 0.75 wt % relative to the total amount of the nutrition composition. More preferably, it is not more than 0.5 wt % relative to the total amount of the nutrition composition. Further preferably, it is not more than 0.3 wt % relative to the total amount of the nutrition composition. Particularly preferably, the nutrition composition of the present invention does not contain isoleucine. When the nutrition composition of the present invention further contains a protein, the above-mentioned isoleucine content includes the amount of isoleucine derived from the protein.

In addition, the content of isoleucine in the nutrition composition of the present invention is not more than 3.8 wt % relative to the total amount of the amino acids, preferably not more than 1.9 wt % relative to the total amount of the amino acids, more preferably not more than 1.5 wt % relative to the total amount of the amino acids, further preferably not more than 1.3 wt % relative to the total amount of the amino acids. Particularly preferably, the nutrition composition of the present invention does not contain isoleucine. When the nutrition composition of the present invention further contains a protein, the above-mentioned isoleucine content includes the amount of isoleucine derived from the protein, and the above-mentioned total amount of the amino acids includes the amount of amino acids derived from the protein.

The amino acid to be used in the nutrition composition of the present invention may be in the form of a salt. Examples of the salt form include a salt with an acid, a salt with a base and the like, with preference given to a pharmaceutically acceptable salt. Examples of such salts include a salt with an inorganic acid, a salt with an organic acid, a salt with an inorganic base, and a salt with an organic base.

Examples of the salt with an inorganic acid include salts with hydrohalic acid (hydrochloric acid, hydrobromic acid, hydroiodic acid etc.), sulfuric acid, nitric acid, phosphoric acid and the like.

Examples of the salt with an organic acid include salts with formic acid, acetic acid, propionic acid, oxalic acid, succinic acid, maleic acid, fumaric acid, citric acid, tartaric acid, methanesulfonic acid, ethanesulfonic acid and the like.

Examples of the salt with an inorganic base include salts with alkali metals such as sodium, potassium and the like, salts with alkaline earth metals such as calcium, magnesium and the like, ammonium salt and the like.

Examples of the salt with an organic base include salts with ethylenediamine, propylenediamine, ethanolamine, monoalkylethanolamine, dialkylethanolamine, diethanolamine, triethanolamine and the like.

The nutrition composition of the present invention suppresses the growth of hemocytozoon.

In the present specification, the hemocytozoon means a parasite living in the blood of the living body and includes, for example, hemosporidia (malaria parasite, leukocytozoon), Wuchereria (filaria), blood fluke (oriental blood fluke), trypanosome (leishmania) and the like. Preferred is a malaria parasite.

In addition, the nutrition composition of the present invention can be used for the treatment of an infectious disease caused by hemocytozoon.

In the present specification, the infectious disease caused by hemocytozoon means a disease caused by infection of hemocytozoon and includes, for example, malaria, filariasis, schistosomiasis, trypanosomiasis and the like. Preferred is malaria.

In the present specification, “the treatment of infectious disease caused by hemocytozoon” includes a reduction of severity of infectious disease caused by hemocytozoon and/or a reduction of mortality rate (that is, an increase of survival rate).

The nutrition composition of the present invention can be used for vertebrates (e.g., chicken, mouse, rat, hamster, rabbit, cat, dog, bovine, horse, sheep, swine, monkey, human etc.). The nutrition composition of the present invention can be preferably used for chickens, monkeys or humans.

The dose of the nutrition composition of the present invention varies depending on the subject of administration, administration route, target disease, symptom and the like. When, for example, the nutrition composition of the present invention is orally ingested by a malaria patient (infant, child, adult), the dose is generally about 100 to about 1000 kcal equivalent, preferably about 150 to about 750 kcal equivalent, which is desirably administered about 1 to several times (e.g., 1 to 6 times) per day for not less than one day and less than 6 months (preferably not less than 2 days and less than 2 months, more preferably not less than 3 days and less than one month, further preferably not less than 4 days and less than 15 days), according to the symptom.

The nutrition composition of the present invention may further contain one or more nutrients selected from the group consisting of carbohydrate, protein, fat, vitamin and mineral. The nutrition composition of the present invention includes a particular nutrition composition containing a particular nutrient (e.g., protein) alone, a complete nutrition diet containing all the above-mentioned nutrients, a therapeutic agent (preferably, antimalarial agent), a comprehensive nutrient, an enteral nutrient, a nutritional supplement, a nutritional treatment food, a dietary supplement, a food with health claims, a supplement and the like.

The nutrition composition of the present invention can be taken orally, transnasally, transesophageally, intragastrically, enterally, intravenously and the like.

The form of the nutrition composition of the present invention is not particularly limited, and can be solid, semi-solid or liquid such as powder (including powder to be dissolved in liquid before intake), tablet, granule, slurry, capsule, solution, jelly, emulsion and the like. In addition, the nutrition composition of the present invention can be served as packed food, packed drink, liquid food, jelly-type food, nutrition bar and the like.

When the nutrition composition of the present invention contains carbohydrate, examples of the carbohydrate source include monosaccharides such as glucose, fructose and the like; disaccharides such as sucrose, malt sugar and the like; oligosaccharides such as fructooligosaccharide, galactooligosaccharide, mannanoligosaccharide, lactosucrose and the like; polysaccharides such as dextrin, dextran, starch, glycogen and the like; sugar alcohols such as xylitol, sorbitol, reduction dextrin, and the like, and the like.

When the nutrition composition of the present invention contains a protein, examples of the protein source include casein and a salt thereof, lactoproteins such as whey and the like, soybean protein, wheat protein, corn protein, potato protein, fish protein, meat protein, egg protein and the like. A protein mainly composed of amino acid is preferable, and corn protein is particularly preferable.

When the nutrition composition of the present invention contains fat, examples of the fat source include plant-derived fats and oils such as avocado oil, flaxseed oil, almond oil, perilla oil, olive oil, cacao butter, grape seed oil, sesame oil, rice oil, safflower oil, Japanese basil oil, soybean oil, camellia oil, evening primrose oil, corn oil, rape seed oil, bran oil, palm oil, palm kernel oil, peanut oil, sunflower oil, hazelnut oil, cottonseed oil, coconut oil, lettuce oil and the like; animal-derived fats and oils such as fish oil, chicken oil, beef fat, lard, mutton tallow and the like; milk fat and processed fats and oils such as butter oil, butter, margarine, and the like, and the like.

When the nutrition composition of the present invention contains a vitamin, examples of the vitamin include liposoluble vitamins such as vitamin A (retinol, retinal, retinoic acid and the like); carotenoid (β-carotene and the like); vitamin D (ergocalciferol, cholecalciferol and the like); vitamin E (α-tocopherol, γ-tocopherol and the like); vitamin K (phylloquinone, menaquinone and the like), water-soluble vitamins such as vitamin B group (vitamin B₁ (thiamine and the like), vitamin B₂ (riboflavin and the like), vitamin B₆ (pyridoxine, pyridoxal, pyridoxamine and the like), vitamin B₁₂ (cyanocobalamin and the like), niacin (nicotinic acid, nicotinic acid amide and the like), pantothenic acid, biotin, folic acid and the like); and vitamin C.

When the nutrition composition of the present invention contains a mineral, examples of the mineral include sodium, potassium, magnesium, calcium, phosphorus, iodine, iron, copper, manganese, selenium, zinc, chrome, molybdenum and the like.

The nutrition composition of the present invention can be used in combination with other antimalarial agent(s). Examples of such other antimalarial agent include quinine hydrochloride, chloroquine, mefloquine, a sulfadoxine/pyrimethamine combination agent, an atovaquone/proguanil combination agent, an artemisinin medicament (artemisinin, artesunate, artemether etc.), an artemether/lumefantrine combination agent, an artesunate/amodiaquine combination agent and the like.

The administration mode for a combined use of the nutrition composition of the present invention and other antimalarial agent(s) is not particularly limited, and the nutrition composition of the present invention and other antimalarial agent(s) only need to be combined on administration. Examples of such administration mode include

(1) administration of a single composition simultaneously containing the nutrition composition of the present invention and other antimalarial agent(s);

(2) simultaneous administration of two kinds of compositions separately containing the nutrition composition of the present invention and other antimalarial agent(s), by the same administration route;

(3) administration of two kinds of compositions separately containing the nutrition composition of the present invention and other antimalarial agent(s), by the same administration route in a staggered manner;

(4) simultaneous administration of two kinds of compositions separately containing the nutrition composition of the present invention and other antimalarial agent(s), by different administration routes; or

(5) administration of two kinds of compositions separately containing the nutrition composition of the present invention and other antimalarial agent(s), by different administration routes in a staggered manner, and the like.

The dose of other antimalarial agent(s) to be used in combination with the nutrition composition of the present invention varies depending on the subject of administration, administration route, symptom and the like. When, for example, an atovaquone/proguanil combination agent is orally ingested as a concomitant drug by a malaria patient (adult), the dose is about 250 mg/100 mg to 1000 mg/400 mg as atovaquone/proguanil hydrochloride for a single ingestion, which is desirably administered about 1 to several times (e.g., 1 to 6 times) per day according to the symptom.

A medicament containing the nutrition composition of the present invention or a medicament simultaneously containing the nutrition composition of the present invention and other antimalarial agent(s) (hereinafter to be referred to as “the medicament of the present invention”) can be formulated into solid agents such as powder, granule, capsule, tablet, chewable agent and the like, liquids such as solution, syrup and the like, or injection, spray and the like by a general method.

In addition, the medicament of the present invention is formulated by adding, depending on the need for formulation, an appropriate pharmaceutically acceptable carrier, for example, excipient, binder, lubricant, solvent, disintegrant, solubilizing agent, suspending agent, emulsifier, isotonic agent, stabilizer, soothing agent, preservative, antioxidant, flavoring agent, colorant and the like. Furthermore, the nutrition composition of the present invention can be prepared together with the above-mentioned carrier.

Examples of the excipient include saccharides such as lactose, glucose, D-mannitol and the like, organic excipients such as starch, cellulose (crystalline cellulose and the like) and the like, inorganic excipients such as calcium carbonate, kaolin, and the like, and the like; examples of the binder include pregelatinized starch, gelatin, gum arabic, methylcellulose, carboxymethylcellulose, sodium carboxymethylcellulose, crystalline cellulose, D-mannitol, trehalose, hydroxypropylcellulose, hydroxypropylmethylcellulose, polyvinylpyrrolidone, polyvinyl alcohol and the like; examples of the lubricant include fatty acid salts such as stearic acid, stearic acid salt and the like, talc, silicate and the like; examples of the solvent include purified water, physiological brine and the like; examples of the disintegrant include low-substituted hydroxypropylcellulose, chemically modified cellulose, starch and the like; examples of the solubilizing agent include polyethylene glycol, propylene glycol, trehalose, benzyl benzoate, ethanol, sodium carbonate, sodium citrate, sodium salicylate, sodium acetate and the like; examples of the suspending agent or emulsifier include sodium lauryl sulfate, gum arabic, gelatin, lecithin, glycerol monostearate, polyvinyl alcohol, polyvinylpyrrolidone, cellulose such as sodium carboxymethylcellulose and the like, polysorbate, polyoxyethylene hydrogenated castor oil and the like; examples of the isotonic agent include sodium chloride, potassium chloride, saccharides, glycerol, urea and the like; examples of the stabilizer include polyethylene glycol, dextran sodium sulfate, other amino acids and the like; examples of the soothing agent include glucose, calcium gluconate, procaine hydrochloride and the like; examples of the preservative include paraoxybenzoate, chlorobutanol, benzyl alcohol, phenethyl alcohol, dehydroacetic acid, sorbic acid and the like; examples of the antioxidant include sulfite, ascorbic acid and the like; examples of the flavoring agent include sweetener, flavor and the like, which are generally used in the fields of medicament and food; examples of the colorant include colorants generally used in the fields of medicament and food.

Other features of the invention will become apparent in the course of the following descriptions of exemplary embodiments which are given for illustration of the invention and are not intended to be limiting thereof.

### EXAMPLES

### Example 1

### Malaria Parasite Growth-Inhibitory Effect in Mice by the Administration of an Isoleucine Deficient Diet and Influence on Survival Rate

As the host animals, BALE/c strain mice (6- to 8-week-old), C3H/HeN strain mice (6- to 8-week-old) and C57BL6/J strain mice (6- to 8-week-old) purchased from CLEA Japan, Inc. were used. The mice were separately bred under the light-dark condition of a 12 hr light period and a 12 hr dark period, given the feed as mentioned below, and allowed to drink water freely. The purchased mice were acclimated for 1 week under the aforementioned light-dark condition, and subjected to the experiment. The feeds given to the animals were powder feeds having the compositions described in the following Table 1. The numerical values in the Table are in % (weight of each component/total weight).

TABLE 1

| | | isoleucine |
|---|---|---|
| | control | deficient |
| | diet | diet |
| histidine | 0.51 | 0.51 |
| isoleucine | 0.89 | 0.00 |
| leucine | 1.63 | 1.63 |
| lysine hydrochloride | 1.76 | 1.76 |
| methionine | 0.49 | 0.49 |
| phenylalanine | 0.90 | 0.90 |
| threonine | 0.76 | 0.76 |
| tryptophan | 0.22 | 0.22 |
| valine | 1.15 | 1.15 |
| alanine | 0.51 | 0.51 |
| arginine | 0.66 | 0.66 |
| asparagine monohydrate | 0.72 | 0.72 |
| aspartic acid | 0.63 | 0.63 |
| L-cystine | 0.40 | 0.40 |
| glutamine | 1.83 | 2.33 |
| glutamic acid | 1.83 | 1.83 |
| glycine | 0.32 | 0.32 |
| proline | 1.87 | 1.87 |
| serine | 1.01 | 1.01 |
| tyrosine | 0.97 | 0.97 |
| cornstarch | 64.18 | 64.58 |
| soybean oil | 7.00 | 7.00 |
| cellulose powder | 5.00 | 5.00 |
| AIN93G mineral mix | 3.50 | 3.50 |
| AIN93 vitamin mix | 1.00 | 1.00 |
| choline bitartrate | 0.25 | 0.25 |
| tertiary butylhydroquinone | 0.0014 | 0.0014 |
| total | 100 | 100 |

Red blood cells infected with the genus Plasmodium were diluted to 1×10⁶/100 μl with phosphate buffer, and intraperitoneally administered to mice 1 week after the start of the ingestion of each feed to infect the mice with a malaria parasite. After infection, a trace amount of blood was collected from the tail edge every day, and a thin layer blood smear specimen was prepared. They were stained with Giemsa, and the ratio of the infected red blood cells to the total red blood cells (parasite-infected red blood cell rate) was calculated by microscopy.

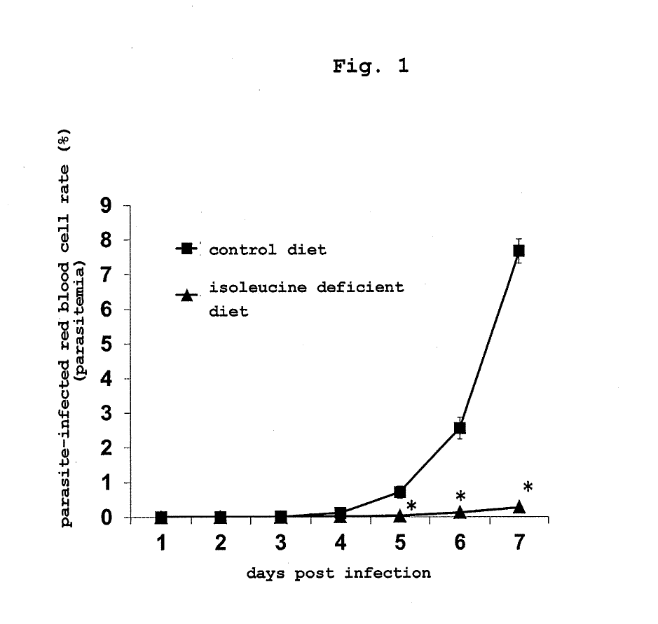

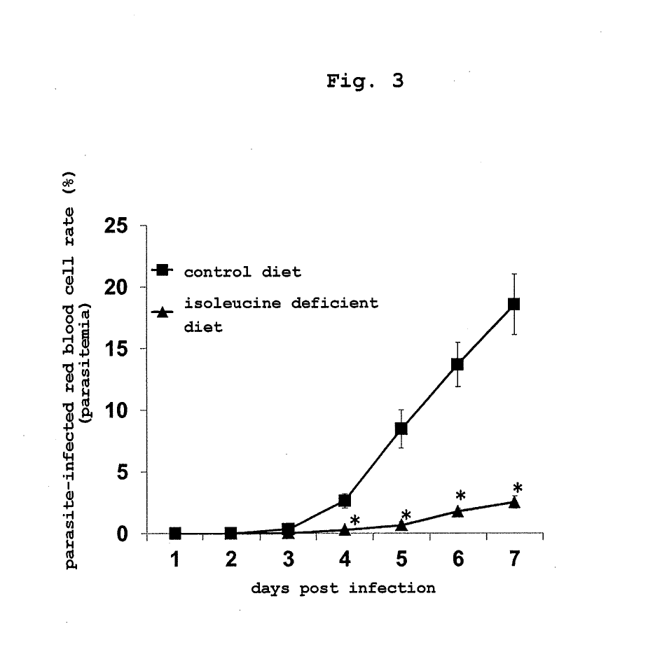

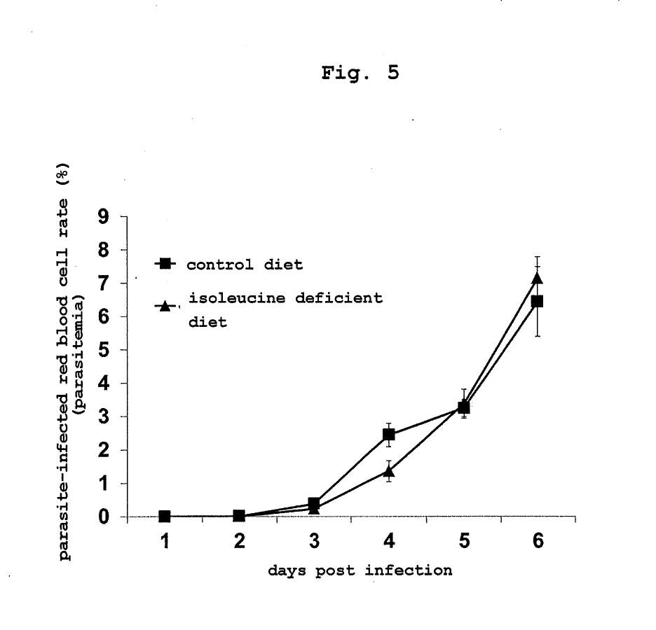

The results of the BALB/c strain mice, C3H/HeN strain mice and C57BL6/J strain mice are shown in FIG. 1, FIG. 3 and FIG. 5, respectively. In FIG. 1, FIG. 3 and FIG. 5, the parasite-infected red blood cell rates are shown by mean±standard error. In addition, 2-way factorial analysis of variance was used for the significance test, and each day was compared according to the Bonferroni method. A critical rate of less than 0.05 was considered to indicate a significant difference from the control group, and is shown with *.

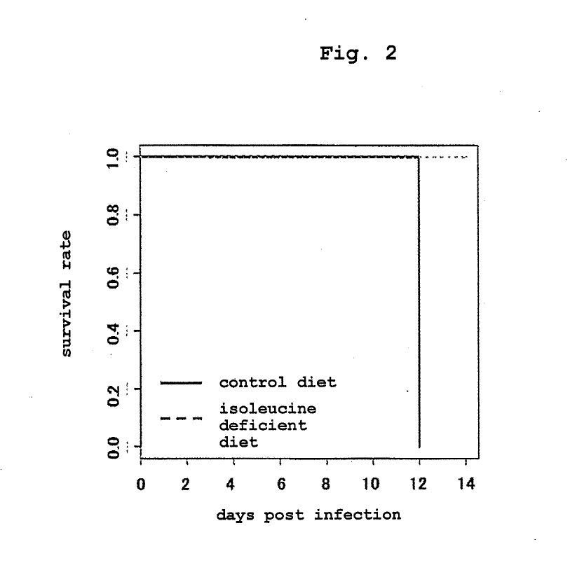

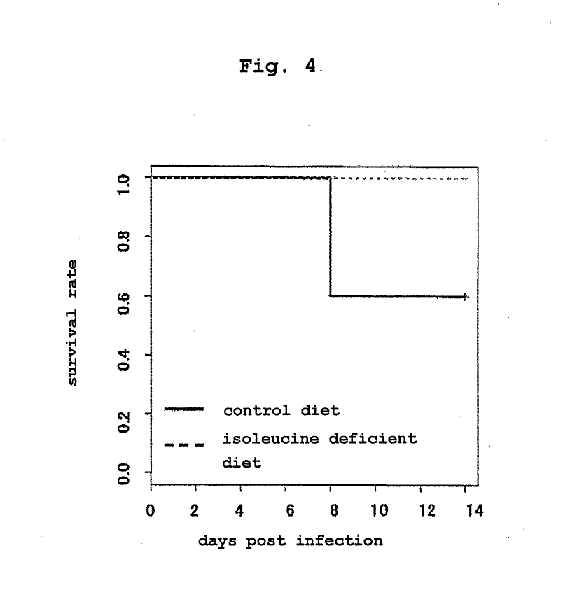

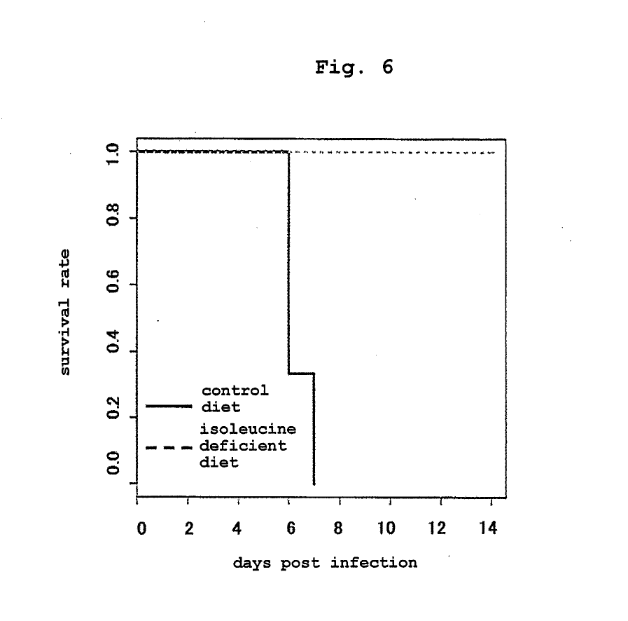

In addition, based on the above-mentioned experiment results, a survival rate curve was drawn using the Kaplan-Meier method. For drawing, R was used (version 2.12.1; R foundation for statistical computing, Vienna, Austria). The survival rate curves of the BALB/c strain mice, C3H/HeN strain mice and C57BL6/J strain mice are shown in FIG. 2, FIG. 4 and FIG. 6, respectively.

In the BALB/c strain mice and C3H/HeN strain mice, the isoleucine deficient diet administration groups showed a strong inhibitory effect on the growth of a malaria parasite (FIG. 1, FIG. 3), and further showed an increase in the malaria survival rate, namely, a life-prolonging effect, as compared to the control group (FIG. 2, FIG. 4).

On the other hand, in the C57BL6/J strain mice, the isoleucine deficient diet administration group did not show a significant inhibitory effect on the growth of a malaria parasite (FIG. 5), but showed an increase in the malaria survival rate, namely, a life-prolonging effect, as compared to the control group (FIG. 6).

### Example 2

### Malaria Parasite Growth-Inhibitory Effect in BALB/c Strain Mice by the Administration of a Diet Having Stepwisely-Decreased Isoleucine Contents

As the host animals, BALB/c strain mice (6- to 8-week-old) purchased from CLEA Japan, Inc. were used. The mice were separately bred under the light-dark condition of a 12 hr light period and a 12 hr dark period, given the feed as mentioned below, and allowed to drink water freely. The purchased mice were acclimated for 1 week under the aforementioned light-dark condition, and subjected to the experiment. The feeds given to the animals were a control diet and diets having a decreased isoleucine content as compared to the control diet (¼ isoleucine-containing diet, ½ isoleucine-containing diet, ¾ isoleucine-containing diet), and powder feeds having the compositions described in the following Table 2 were used. The numerical values in the Table are in % (weight of each component/total weight).

TABLE 2

| | | ¼ | ½ | ¾ |
|---|---|---|---|---|
| | | isoleucine- | isoleucine- | isoleucine- |
| | control | containing | containing | containing |
| | diet | diet | diet | diet |
| histidine | 0.51 | 0.51 | 0.51 | 0.51 |
| isoleucine | 0.89 | 0.22 | 0.44 | 0.67 |
| leucine | 1.63 | 1.63 | 1.63 | 1.63 |
| lysine hydrochloride | 1.76 | 1.76 | 1.76 | 1.76 |
| methionine | 0.49 | 0.49 | 0.49 | 0.49 |
| phenylalanine | 0.90 | 0.90 | 0.90 | 0.90 |
| threonine | 0.76 | 0.76 | 0.76 | 0.76 |
| tryptophan | 0.22 | 0.22 | 0.22 | 0.22 |
| valine | 1.15 | 1.15 | 1.15 | 1.15 |
| alanine | 0.51 | 0.51 | 0.51 | 0.51 |
| arginine | 0.66 | 0.66 | 0.66 | 0.66 |
| asparagine | 0.72 | 0.72 | 0.72 | 0.72 |
| monohydrate | | | | |
| aspartic acid | 0.63 | 0.63 | 0.63 | 0.63 |
| L-cystine | 0.40 | 0.40 | 0.40 | 0.40 |
| glutamine | 1.83 | 2.20 | 2.08 | 1.96 |
| glutamic acid | 1.83 | 1.83 | 1.83 | 1.83 |
| glycine | 0.32 | 0.32 | 0.32 | 0.32 |
| proline | 1.87 | 1.87 | 1.87 | 1.87 |
| serine | 1.01 | 1.01 | 1.01 | 1.01 |
| tyrosine | 0.97 | 0.97 | 0.97 | 0.97 |
| cornstarch | 64.18 | 64.48 | 64.38 | 64.28 |
| soybean oil | 7.00 | 7.00 | 7.00 | 7.00 |
| cellulose powder | 5.00 | 5.00 | 5.00 | 5.00 |
| AIN93G mineral mix | 3.50 | 3.50 | 3.50 | 3.50 |
| AIN93 vitamin mix | 1.00 | 1.00 | 1.00 | 1.00 |
| choline bitartrate | 0.25 | 0.25 | 0.25 | 0.25 |
| tertiary | 0.0014 | 0.0014 | 0.0014 | 0.0014 |
| butylhydroquinone | | | | |
| total | 100 | 100 | 100 | 100 |

Red blood cells infected with the genus Plasmodium were diluted to 1×10⁶/100 μl with phosphate buffer, and intraperitoneally administered to mice 1 week after the start of the ingestion of each feed to infect the mice with a malaria parasite. After infection, a trace amount of blood was collected from the tail edge every day, and a thin layer blood smear specimen was prepared. They were stained with Giemsa, and the ratio of the infected red blood cells to the total red blood cells (parasite-infected red blood cell rate) was calculated by microscopy.

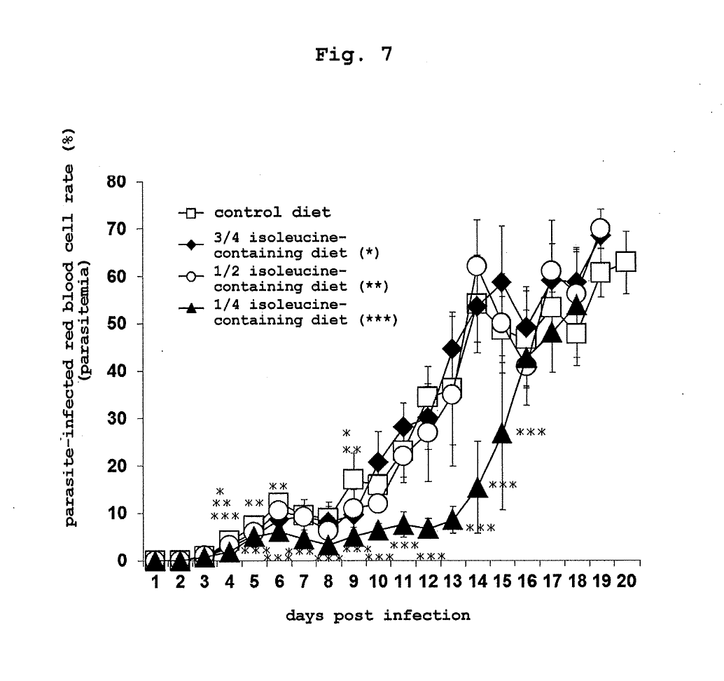

The results are shown in FIG. 7. In FIG. 7, the parasite-infected red blood cell rates are shown by mean±standard error. In addition, 2-way factorial analysis of variance was used for the significance test, and each day was compared according to the Bonferroni method. A critical rate of less than 0.05 was considered to indicate a significant difference from the control group, and is shown with * (significant difference between control group and ¾ isoleucine-containing diet administration group), ** (significant difference between control group and ½ isoleucine-containing diet administration group), and *** (significant difference between control group and ¼ isoleucine-containing diet administration group).

The control group and ¾ and ½ isoleucine-containing diet administration groups did not show a malaria parasite growth inhibitory effect, whereas the ¼ isoleucine-containing diet administration group showed a malaria parasite growth inhibitory effect.

The above results reveal that the growth of a malaria parasite is inhibited when the content of isoleucine in the diet is not more than a certain level.

### Example 3

### Malaria Parasite Growth-Inhibitory Effect in BALB/c Strain Mice by the Administration of an Isoleucine Deficient Diet after Infection with a Malaria Parasite

As the host animals, BALE/c strain mice (6- to 8-week-old) purchased from CLEA Japan, Inc. were used. The mice were separately bred under the light-dark condition of a 12 hr light period and a 12 hr dark period, and allowed to take feed and water freely. The purchased mice were acclimated for 1 week under the aforementioned light-dark condition, and subjected to the experiment. The feeds given to the animals were powder feeds having the compositions described in Table 1 of Example 1.

Red blood cells infected with the genus Plasmodium were diluted to 1×10⁶/100 μl with phosphate buffer, and intraperitoneally administered to mice 1 week after the start of the ingestion of the control feed to infect the mice with a malaria parasite. One week after the intraperitoneal administration, the mice were divided into a group to be fed with an isoleucine deficient diet and a group to be continuously fed with the control diet. After infection, moreover, a trace amount of blood was collected from the tail edge every day, and a thin layer blood smear specimen was prepared. They were stained with Giemsa, and the ratio of the infected red blood cells to the total red blood cells (parasite-infected red blood cell rate) was calculated by microscopy.

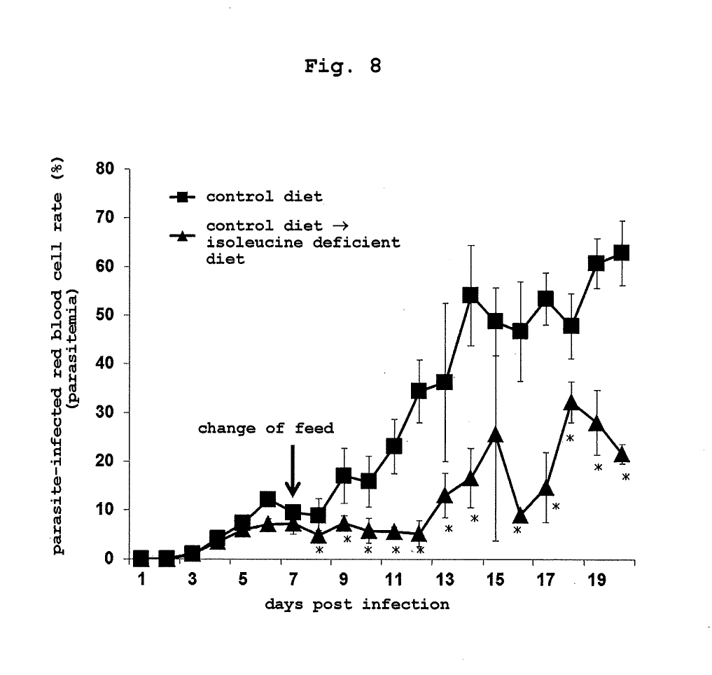

The results are shown in FIG. 8. In FIG. 8, the parasite-infected red blood cell rates are shown by mean±standard error. In addition, 2-way factorial analysis of variance was used for the significance test, and each day was compared according to the Bonferroni method. A critical rate of less than 0.05 was considered to indicate a significant difference from the control group, and is shown with *.

The experiment group, wherein the diet to be fed was changed from the control diet to the isoleucine deficient diet 2 weeks from the start of the administration, showed a malaria parasite growth inhibitory effect. Therefore, it was shown that ingestion of an isoleucine deficient diet can inhibit the growth of a malaria parasite even after infection with the malaria parasite.

### INDUSTRIAL APPLICABILITY

The nutrition composition of the present invention can suppress the growth of hemocytozoon such as malaria parasite and the like. In addition, the nutrition composition of the present invention can be used for the treatment of infectious disease caused by hemocytozoon such as malaria. Furthermore, the nutrition composition of the present invention is superior in safety, since the ingredients thereof are amino acids and the like, which are less feared to cause side effects.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A nutrition composition, comprising:
(a) tryptophan, methionine, phenylalanine, valine, leucine, histidine, lysine, and threonine; and
(b) optionally isoleucine, wherein when said isoleucine is present, it is present in an amount of not more than 1.5 wt % relative to the total amount of the nutrition composition.

2. A nutrition composition, comprising:
(a) tryptophan, methionine, phenylalanine, valine, leucine, histidine, lysine, and threonine; and
(b) optionally isoleucine, wherein when said isoleucine is present, it is present in an amount of not more than 3.8 wt % relative to the total amount of the amino acids.

3. The nutrition composition according to claim 1, which does not contain isoleucine.

4. The nutrition composition according to claim 2, which does not contain isoleucine.

5. The nutrition composition according to claim 1, further comprising one or more nutrients selected from the group consisting of carbohydrate, protein, fat, vitamin and mineral.

6. The nutrition composition according to claim 2, further comprising one or more nutrients selected from the group consisting of carbohydrate, protein, fat, vitamin and mineral.

7. A method of suppressing the growth of a hemocytozoon, comprising administering, to a vertebrate in need thereof, an effective amount of a nutrition composition comprising:
(a) one or more amino acids selected from the group consisting of tryptophan, methionine, phenylalanine, valine, leucine, histidine, lysine, and threonine; and
(b) optionally isoleucine, wherein when said isoleucine is present, it is present in an amount of not more than 1.5 wt % relative to the total amount of the nutrition composition.

8. The method according to claim 7, wherein the nutrition composition does not contain isoleucine.

9. The method according to claim 7, further comprising one or more nutrients selected from the group consisting of carbohydrate, protein, fat, vitamin and mineral.

10. The method according to claim 7, wherein the hemocytozoon is a malaria parasite.

11. The method according to claim 7, further comprising administering an effective amount of other antimalarial agent(s).

12. The method according to claim 11, wherein said other antimalarial agent is one or more medicaments selected from the group consisting of quinine hydrochloride, chloroquine, mefloquine, a sulfadoxine/pyrimethamine combination agent, an atovaquone/proguanil combination agent, artemisinin, artesunate, artemether, an artemether/lumefantrine combination agent and an artesunate/amodiaquine combination agent.

13. A method of suppressing the growth of a hemocytozoon, comprising administering, to a vertebrate in need thereof, an effective amount of a nutrition composition comprising:
(a) one or more amino acids selected from the group consisting of tryptophan, methionine, phenylalanine, valine, leucine, histidine, lysine, and threonine; and
(b) optionally isoleucine, wherein when said isoleucine is present, it is present in an amount of not more than 3.8 wt % relative to the total amount of the amino acids.

14. The method according to claim 13, wherein the nutrition composition does not contain isoleucine.

15. The method according to claim 13, further comprising one or more nutrients selected from the group consisting of carbohydrate, protein, fat, vitamin and mineral.

16. The method according to claim 13, wherein the hemocytozoon is a malaria parasite.

17. The method according to claim 13, further comprising administering an effective amount of other antimalarial agent(s).

18. The method according to claim 17, wherein said other antimalarial agent is one or more medicaments selected from the group consisting of quinine hydrochloride, chloroquine, mefloquine, a sulfadoxine/pyrimethamine combination agent, an atovaquone/proguanil combination agent, artemisinin, artesunate, artemether, an artemether/lumefantrine combination agent and an artesunate/amodiaquine combination agent.

19. A method of treating infectious disease caused by hemocytozoon, comprising administering, to a vertebrate in need thereof, an effective amount of a nutrition composition comprising:
(a) one or more amino acids selected from the group consisting of tryptophan, methionine, phenylalanine, valine, leucine, histidine, lysine, and threonine; and
(b) optionally isoleucine, wherein when said isoleucine is present, it is present in an amount of not more than 1.5 wt % relative to the total amount of the nutrition composition.

20. The method according to claim 19, wherein the nutrition composition does not contain isoleucine.

21. The method according to claim 19, further comprising one or more nutrients selected from the group consisting of carbohydrate, protein, fat, vitamin and mineral.

22. The method according to claim 19, wherein the hemocytozoon is a malaria parasite.

23. The method according to claim 19, further comprising administering an effective amount of other antimalarial agent(s).

24. The method according to claim 23, wherein said other antimalarial agent is one or more medicaments selected from the group consisting of quinine hydrochloride, chloroquine, mefloquine, a sulfadoxine/pyrimethamine combination agent, an atovaquone/proguanil combination agent, artemisinin, artesunate, artemether, an artemether/lumefantrine combination agent and an artesunate/amodiaquine combination agent.

25. A method of treating infectious disease caused by hemocytozoon, comprising administering, to a vertebrate in need thereof, an effective amount of a nutrition composition comprising:
(a) one or more amino acids selected from the group consisting of tryptophan, methionine, phenylalanine, valine, leucine, histidine, lysine, and threonine; and
(b) optionally isoleucine, wherein when said isoleucine is present, it is present in an amount of not more than 3.8 wt % relative to the total amount of the amino acids.

26. The method according to claim 25, wherein the nutrition composition does not contain isoleucine.

27. The method according to claim 25, further comprising one or more nutrients selected from the group consisting of carbohydrate, protein, fat, vitamin and mineral.

28. The method according to claim 25, wherein the hemocytozoon is a malaria parasite.

29. The method according to claim 25, further comprising administering an effective amount of other antimalarial agent(s).

30. The method according to claim 29, wherein said other antimalarial agent is one or more medicaments selected from the group consisting of quinine hydrochloride, chloroquine, mefloquine, a sulfadoxine/pyrimethamine combination agent, an atovaquone/proguanil combination agent, artemisinin, artesunate, artemether, an artemether/lumefantrine combination agent and an artesunate/amodiaquine combination agent.
